(19) United States
(12) Patent Application Publication
(10) Pub. No.: US 2018/0185772 A1
(43) Pub. Date: Jul. 5, 2018

(54) CENTRIFUGE AND SYSTEM FOR BODILY FLUID SAMPLE

(71) Applicants: Marko Karhiniemi, Helsinki (FI); Mika Karilahti, Helsinki (FI); Medigoo Oy, Espoo (FI)

(72) Inventors: Marko Karhiniemi, Helsinki (FI); Mika Karilahti, Helsinki (FI)

(21) Appl. No.: 15/739,520

(22) PCT Filed: Jun. 21, 2016
(86) PCT No.: PCT/FI2016/050449

§ 371 Date: Dec. 22, 2017

(30) Foreign Application Priority Data
Jun. 23, 2015 (GB) 1511039.8

(51) Int. Cl. B01D 21/26; B01L 3/00; B04B 1/02; B04B 5/04; B04B 7/08; B04B 9/04; G01N 33/49

(52) U.S. Cl. CPC B01D 21/262; B01L 3/5021; B04B 1/02; B04B 5/0407; B01L 2300/123; B04B 9/04; G01N 33/491; B01L 2300/0851; B01L 2300/0858; B04B 7/08

(57) ABSTRACT

The invention relates generally to health tests and needs to examine bodily fluids such as blood. The invention comprises a centrifuge and system for a bodily fluid sample. The centrifuge comprises a sample chamber for receiving the bodily fluid sample through an opening so that the bodily fluid sample rests against an inner surface of the sample chamber. The centrifuge further comprises a contact part for coupling the sample chamber to an actuator, the actuator being configured to rotate the sample chamber and cause sedimentation of the bodily fluid sample.

### TECHNICAL FIELD

The present application relates generally to health tests and needs to examine bodily fluids, especially blood samples.

### BACKGROUND OF THE INVENTION

In the past the most medical tests were performed in medical laboratories, which entailed sending specimens from a point of care and then waiting hours or days the results. It is forecasted that the in-vitro-diagnostics (IVD) and point-of-care (POC) testing are driving the health care markets. Another driving force is the importance of getting medical results faster. Many IVD tests can be used at the POC, or they are patient self-tests, and some of them are rapid tests that give the results in a few minutes.

In vivo refers to diagnostic tests conducted within a living organism. Conversely, in-vitro-diagnostics refers to tests that are conducted outside a living body. A blood glucose test is an example of the IVD test intended for patients with diabetes. Other IVD test examples are: a pregnancy test, an infectious disease test, and an STD (sexually transmitted disease) test.

Different bodily fluid samples are needed for IVD tests. The most common are blood or urine samples. For example, blood sampling is performed for tests or other procedures. Generally, blood sampling include arterial blood sampling, such as by radial artery puncture, capillary blood sampling (generally using a lancet), and venous blood sampling in which blood samples are collected in capped test tubes.

Numerous IVD tests are based on serum/plasma and the separation of whole blood into its components is required. Centrifugation is a well-known method by which plasma and serum is separated. The centrifugation is time consuming and requires heavy laboratory equipment. Additionally, the further processing and analysis requires multiple steps where additional laboratory tasks are required. A drawback of the prior art is that centrifuges are expensive and slow from a point of view POC testing and self-testing.

Various methods and devices for blood separation have been presented over the years. Diane L. et al, discusses many of these in U.S. Pat. No. 4,933,092. For example, a layer of glass fibres can be used to separate the blood components. Blood separation devices utilizing glass fibre membranes tend to separate serum at a relatively slow speed. Another drawback is that the glass fibre retains significant quantities of serum or plasma in the interstices of the membrane, which causes that the amount of a blood sample should relatively large.

Regarding the specimen collection, bodily fluid analysation, and/or blood separation US2014/273187 discusses point of care sensor systems that include a portable reader and a disposable cartridge with a membrane or filter, WO2003/060479 discusses a sample testing device, buffer fluid, and a filter for holding a test strip, EP2684607 discusses filters and fluid analysis cartridges, and US2005/232813 discusses filters and capillary tubes, and WO2007/075922 discusses a portable sample analyser that includes a disposable fluidic cartridge.

### SUMMARY OF THE INVENTION

An objective of the invention is an apparatus for a fast blood component separation. Another objective of the invention is that the serum or plasma included in a blood sample can be utilized in point-of-care testing. Another objective of the invention is a small-sized, easy-to-use, and/or cost-effective centrifuge and the centrifuge-based system for self-tests and other (health) tests. In addition to blood, the tests may be based on other bodily fluids: saliva, urine, perspiration, tears, sperm, or interstitial fluid.

The invention concerns a centrifuge for a bodily fluid sample. A single bodily fluid sample can be immediately tested when it is obtained. The centrifuge comprises

a sample chamber for receiving the bodily fluid sample through an opening so that the bodily fluid sample rests against an inner surface of the sample chamber and

a contact part for coupling the sample chamber to an actuator, the actuator being configured to rotate the sample chamber and cause a sedimentation of the bodily fluid sample.

In one embodiment the sample chamber includes a sedimentation basin for gathering the heaviest component of the bodily fluid sample when the actuator rotates the sample chamber, the sedimentation basin locating on the utmost surface of the sample chamber from an axis of rotation.

In one embodiment the sample chamber comprises a flexible material so that a distance between the sedimentation basin and the axis of rotation can vary.

In one embodiment the sample chamber includes a bottom basin into which the gravitation forces the heaviest component of the bodily fluid sample when a rotation of the sample chamber has stopped.

In one embodiment the centrifuge comprises a stopper on the opening.

In one embodiment the sample chamber comprises a transparent material to provide a view inside the sample chamber.

In one embodiment the sample chamber comprises a coating on its inner surface.

In one embodiment the contact part includes threads.

In one embodiment the centrifuge comprises the actuator.

In one embodiment the actuator comprises at least one of the following parts: an electric motor, a loadable accumulator.

The invention further concerns a system for a bodily fluid sample. The system includes such centrifuge that comprises

a sample chamber for receiving the bodily fluid sample through an opening so that the bodily fluid sample rests against an inner surface of the sample chamber and

a contact part for coupling the sample chamber to an actuator, the actuator being configured to rotate the sample chamber and cause a sedimentation of the bodily fluid sample.

In one embodiment the system comprises one of the following parts for moving the bodily fluid sample or its component: a pipette, a capillary pipe, an injection.

In one embodiment the system comprises a test cassette.

In one embodiment the test cassette comprises

a well for receiving a component of the bodily fluid sample to be tested and

an indicator for showing a test result.

In one embodiment at least one of the following parts is a fixed part of the system: a lancet for taking a blood sample, the test cassette, a part for moving the bodily fluid sample or its component.

### BRIEF DESCRIPTION OF THE DRAWINGS

For a more complete understanding of examples and embodiments of the present invention, reference is now made to the following descriptions taken in connection with the accompanying drawings in which:

FIG. 1 shows a cross-section of a cone type of centrifuge,

FIG. 2 shows a centrifuge from a bird's view,

FIG. 3 shows a cross-section of a centrifuge when it is in use,

FIG. 4 shows a cross-section of a disposable centrifuge,

FIG. 5 shows a propeller type of centrifuge from a bird's view and a cross-section of the centrifuge,

FIG. 6 shows a cross-section of a centrifuge comprising an actuator,

FIG. 7 shows a nomogram for a centrifuge,

FIG. 8 shows a system comprising a centrifuge.

### DETAILED DESCRIPTION OF THE INVENTION

It is appreciated that the following embodiments are exemplary. Although the specification may refer to “one” or “some” embodiment(s), the reference is not necessarily made to the same embodiment(s), or the feature in question may apply to multiple embodiments. Single features of different embodiments may be combined to provide further embodiments.

Let us assume that there is a motor or an actuator to provide the required circulation force to a separator. There may be a vent hole, i.e. an opening, on the side of the separator to insert fluid to the separator and/or take the separated fluid, such as blood component, out of the separator. Alternatively the vent hole may be on the center of the separator. The separator can be also termed a centrifuge.

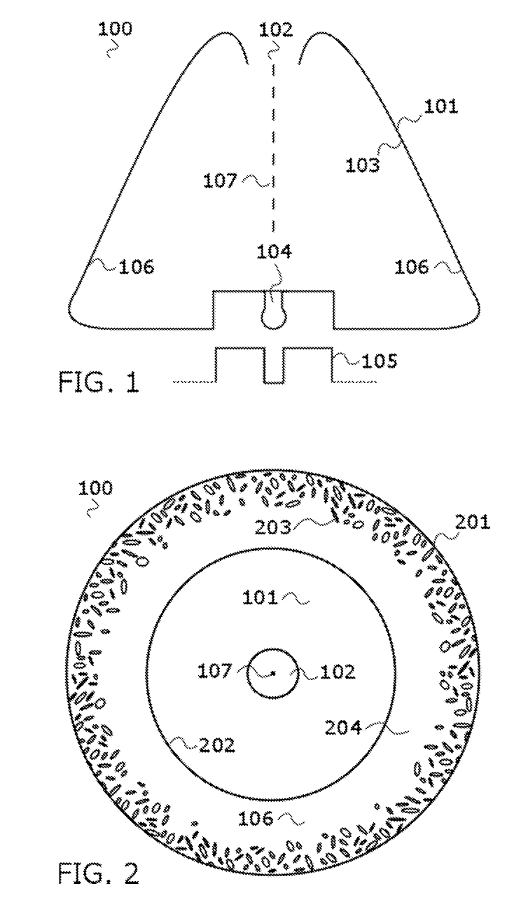

FIG. 1 shows a cross-section of a cone type of centrifuge. The cone, or a cut cone, is appropriate form but not the only possible form for a centrifuge 100. Centrifuge 100 is configured to separate a component of a bodily fluid sample. Blood is an example of the bodily fluid sample and serum/plasma is an example of the component. Generally speaking, centrifuge 100 is intended for at least one of the following bodily fluid samples: blood, saliva, urine, perspiration, tears, sperm, or interstitial fluid.

Centrifuge 100 comprises a sample chamber 101 for receiving the bodily fluid sample through an opening 102 so that the bodily fluid sample touches an inner surface 103 of sample chamber 101 and stays rest against inner surface 103 of the sample chamber. This feature differentiates centrifuge 100 from the prior art centrifuges that are configured to receive SST tubes (serum-separating tubes) or other sample tubes inside them.

Centrifuge 100 may be small-sized and its diameter is, for example, two cm. Centrifuge 100 is intended for a single bodily fluid sample. It is possible to receive the sample to centrifuge 100 immediately after taking the sample.

Centrifuge 100 further comprises a contact part 104 for coupling sample chamber 101 to an actuator 105. Contact part 104 may be a hole in centrifuge 100. In FIG. 1 contact part 104 is a protrusion that fits into a counterpart of actuator 105 and a friction keeps it in the counterpart.

Actuator 105 includes, for example, an electric motor for rotating its axle and the counterpart attached to the axle ten thousands rounds per minute. Actuator 105 may be a hand-hold apparatus that can rotate sample chamber 101 fast enough about contact part 104 to cause the sedimentation in the bodily fluid sample.

Centrifuge 100 further comprises a sedimentation basin 106 for gathering the heaviest component of the body fluid sample when actuator 105 is coupled to contact part 104 and rotates sample chamber 101. The heaviest component has the greatest mass per unit volume compared to the other component(s) included in the body fluid sample. The rotation of sample chamber 101 causes the sedimentation that separates the heaviest component, such as blood cells, from the bodily fluid sample. Sedimentation basin 106 locates inside sample chamber 101 on the utmost surface from an axis of rotation 107. Axis of rotation 107 is illustrated with a dashed line in FIG. 1.

In the embodiment shown in FIG. 1 the distance between sedimentation basin 106 and axis of rotation 107 is the same when centrifuge 100 rotates or is stopped.

Contact part 104 is positioned in relation to sample chamber 101 so that axis of rotation 107 penetrates it and sample chamber 101 and enables a rotation of sample chamber 101 in a balanced manner. As the prior art centrifuges, centrifuge 100 must be rotatable in the balanced manner to ensure its safe use and a successful separation of the components of the bodily fluid sample.

FIG. 2 shows a cross-section of centrifuge 100 from a bird's view when sample chamber 101 is rotating the full speed. Sample chamber 101 has a shape of a circle from this perspective. A dot in the middle is axis of rotation 107 and the smallest circle around the axis of rotation 107 is opening 102 of centrifuge 100.

A circle between the utmost circumference 201 of sample chamber 101 and opening 102 represents a surface 202 of a blood sample. The centrifugal force affects at the full speed so that the blood sample is located in sedimentation basin 106. Sedimentation basin 106 is the utmost surface from axis of rotation 107 and it can be considered as a portion of the inner surface of sample chamber 101 between opening 102 and the utmost circumference 201 of sample chamber 101.

Ovals in sample chamber 101 represent blood cells 203 which are ended due to the sedimentation into sedimentation basin 106. Blood cells 203 form such component of the blood sample that has a greater mass per unit volume than the rest portion of the blood sample. The rest portion of the blood sample, i.e. plasma 204, is located between blood cells 203 and surface 202 of the blood sample. Basically, it is possible to use centrifuge 100 without a stopper on opening 102, if the amount of the bodily fluid sample is small enough. In more detail, the stopper could be omitted, if surface 202 of the bodily fluid sample does not reach opening 102. The stopper is, however, useful for safety reasons.

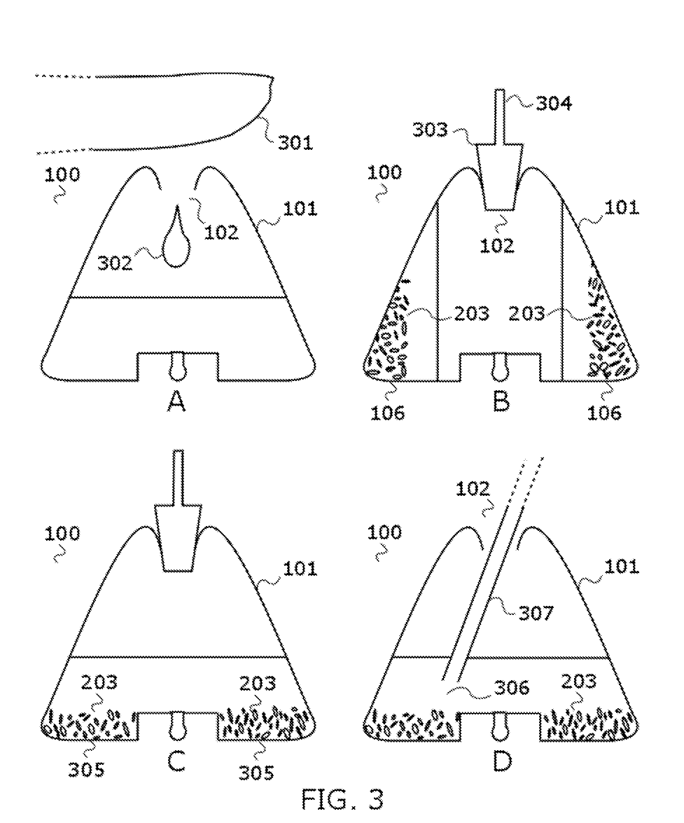

FIG. 3 shows a use of centrifuge 100 in four views from ‘A’ to ‘D’.

View ‘A’ illustrates receiving a blood sample in sample chamber 101. A user's finger 301 is located on a top of opening 102 and a blood drop 302. Sample chamber 101 is not rotating and thus the blood sample is located on the bottom of sample chamber 101.

View ‘B’ illustrates a situation in which opening 102 is closed with a stopper 303 to prevent a spilling of the blood sample and sample chamber 101 is rotating at the full speed. Stopper 303 is, for example, a plug with a little rod 304 to grip with fingers. Blood cells 203 are ended due to the sedimentation into sedimentation basin 106, i.e. to the sides of sample chamber 101.

View ‘C’ illustrates a situation in which centrifuge 100 is stopped and, due to the gravitation, blood cells 203 are dropped to the bottom of sample chamber 101. In this embodiment the bottom part of sample chamber 101 operates as a bottom basin 305 for storing the component. Blood cells 203 end into bottom basin 305 when the sedimentation has reached a predetermined level and centrifuge 100 is stopped.

View ‘D’ illustrates taking plasma 306 from sample chamber 101. A person has removed stopper 303 and pushed the end of a pipette 307 through opening 102 into plasma 306. In one embodiment centrifuge 100 comprises a transparent material to provide a view into centrifuge 100. In one embodiment centrifuge 100 is made of transparent material so that the person taking plasma 306 from sample chamber 101 can see through centrifuge 100 the upper surface of plasma 306 and the boundary surface between plasma 306 and blood cells 203 and therefore the person can target the end of pipette 307 into plasma 306.

It is possible to provide such air pressure to the end of pipette 307 that plasma 306 does not enter into pipette 307. When the end of pipette 307 is in the layer of blood cells 203 the person can stop squeezing the pipette after which he/she can take blood cells 203 into the pipette. Thus, the invention is not limited to the use of the lightest layer of the bodily fluid sample but the other layer(s) of the sample can be examined, too.

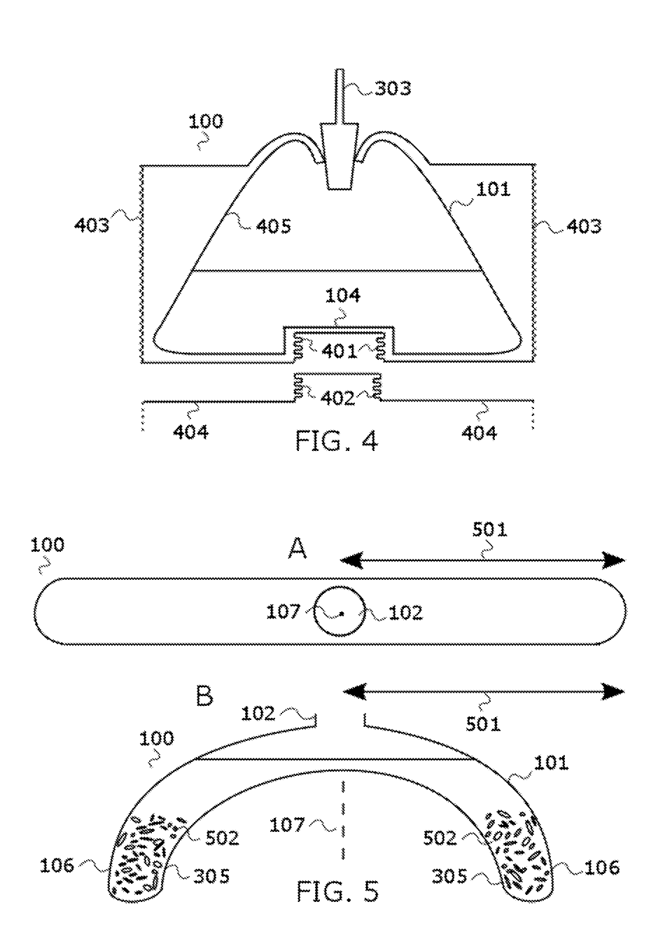

FIG. 4 shows a cross-section of a disposable centrifuge. Disposable centrifuge 100 is intended to be used once or less than ten times. In this embodiment centrifuge 100 has a size of a bottle cap and contact part 104 includes threads 401, just like a bottle cap. Also actuator 105 includes threads 402 to which threads 401 of contact part 104 can be screwed. Like in bottle caps, surface 403 of centrifuge 100 is made rough. In addition, actuator 105 comprises a disc shaped part having a rough surface 404.

The rough surfaces 403, 404 makes easier to attach centrifuge 100 to actuator 105 (to the counterpart of actuator 105) and to detach it. In one embodiment centrifuge 100 is made of plastic or glass and it can be disposed with the blood sample when a component to be analysed is taken from the blood sample. Stopper 303 keeps the sample inside the centrifuge 100. Stopper 303 further prevents the blood sample from drying when actuator 105 rotates centrifuge 100. Another purpose for stopper 303 is that it remains sample chamber 101 sterile from the manufacturing of centrifuge 100 to usage of it. Still another purpose for stopper 303 is that it keeps a special gel (for blood) in sample chamber 101.

In one embodiment sample chamber 101 comprises a coating 405 on its inner surface. One purpose for coating 405 is to prevent the chemicals of plastic to react with the bodily fluid sample and contaminate the sample, if sample chamber 101 is made of plastic. Another purpose for coating 405 is to prevent a clotting of blood.

Still another purpose for coating 405 of the inner surface of sample chamber 101 is to prevent the (blood) sample to grip to the inner surface of sample chamber 101, because the amount of the sample may be tiny, only few drops, and thus the sample should be carefully utilized to obtain reliable test results. Teflon may be an appropriate substance to be used as slippery coating 405. A layer of liquid perfluorocarbon is another substance to reduce the friction of coating 405, especially TLP (tethered-liquid perfluorocarbon) developed at Harvard School of Engineering and Applied Sciences (SEAS).

The centrifuge 100 is configured to receive the bodily fluid sample into sample chamber 101. When centrifuge 100 comprises coating 405, coating 405 operates as the inner surface of sample chamber 101 against which the bodily fluid sample rests.

In one embodiment centrifuge 100 is reusable. In more detail, centrifuge 100 and its sample chamber 101 are cleanable with a liquid. The liquid may be pure water or it may contain a cleaning/sterilizing substance.

FIG. 5 shows a propeller type of centrifuge. View ‘A’ shows centrifuge 100 from a bird's view. In this embodiment centrifuge 100 is relatively long and thin and it looks a propeller. Centrifuge 100 is shaped so that it can rotate on axis of rotation 107 as fast as possible. Therefore, conversely as a propeller, centrifuge 100 does not aim to affect the ambient air. An advantage of the propeller type of centrifuge 100 is a longer radius of rotation 501 and stronger centrifugal force compared to the cone type of centrifuge 100 shown in Fig. A. An advantage of the cone type of centrifuge 100 is it has a lower air resistance and thus higher rotation speed than the propeller type of centrifuge 100.

View ‘B’ shows a cross-section of the propeller type of centrifuge 100 from its side. In view ‘B’ the sedimentation of a bodily fluid sample has happened and centrifuge 100 is stopped. The heaviest component 502 of the bodily fluid sample has ended due to the gravity into bottom basin 305. As shown in view ‘B’, the ends of the propeller type of centrifuge 100 are twisted downwards (towards the earth) and operate as bottom basin 305. Ovals represent the heaviest component 502.

View ‘B’ illustrates an embodiment in which sample chamber 101 comprises a flexible or bendable material. Due to the flexible or bendable material a distance between the sedimentation basin and the axis of rotation can vary. In more detail, a rotation of sample chamber 101 causes sedimentation basin 106 to recede from axis of rotation 107. An advantage of the flexible sample chamber 101 is that sample chamber 101 has a compact size when it is not rotated and sample chamber 101 is long, and thus radius of rotation 501 is long, when sample chamber 101 is rotated (the long radius increases the effectiveness of the sedimentation).

View ‘B’ is also an example of sample chamber 101 in which sedimentation basin 106 and bottom basin 305 are basically the same basin (in FIG. 3 sedimentation basin 106 and bottom basin 305 are basins though they partly overlap).

In addition to the cone type of centrifuge 100 shown in FIG. 1 and the propeller type of centrifuge 100 shown in FIG. 5, other centrifuge forms are possible, too. In one embodiment centrifuge 100 has a form of cylinder. In one embodiment centrifuge 100 has a form of disc. Centrifuge 100 can be formed in various manners so that the utmost surface of sample chamber 101 from an axis of rotation 107 operates as sedimentation basin 106 when actuator 105 (not shown) rotates centrifuge 100.

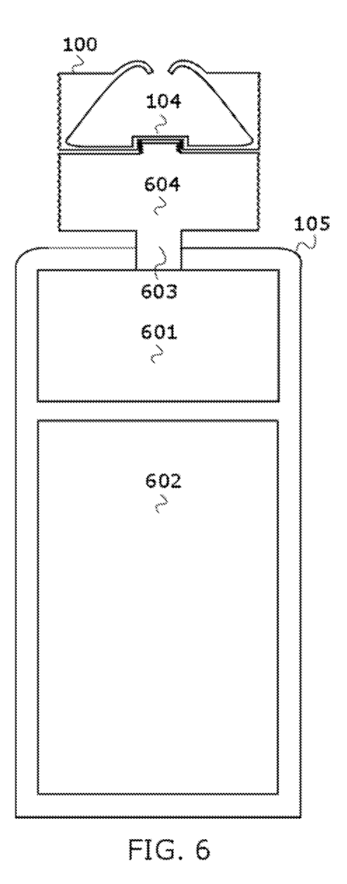

FIG. 6 shows a cross-section of centrifuge 100 that comprises actuator 105 equipped with an electric motor 601. Actuator 105 is size of an electric toothbrush and has a similar a loadable accumulator 602. Electric motor 601 can rotate an axle 603 that is firmly attached to a counterpart 604. Counterpart 604 is detachable attached (with threads) to contact part 104 of centrifuge 100.

The components of the bodily fluid sample may remix together, if centrifuge 100 stops too rapidly after the sedimentation. Therefore, in one embodiment of centrifuge 100 the rotational speed of actuator 105 is adjustable to avoid a remixing of the components. This feature can be implemented by using a motor control, for example, a pulse-width modulation (PWM). The motor control is advantageous, if the components of the bodily fluid sample will easily remix. If the remixing does not happen easily, actuator 105 can be an on/off type of actuator. When actuator 105 is turned from the on state to the off state, the friction finally stops the rotation of centrifuge 100. It is recommendable to hold centrifuge 100 and actuator 105 on a horizontal plane (a table) to avoid the remixing.

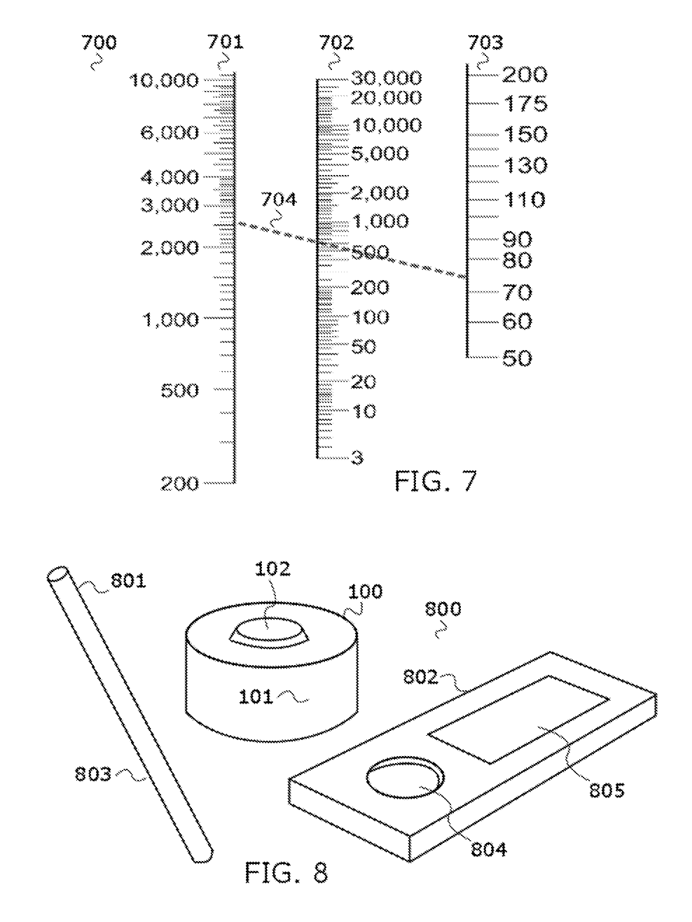

FIG. 7 shows a nomogram 700 for centrifuge 100. Nomogram 700 comprises a scale 701 for RFC, a scale 702 for RPM, and a scale 703 for Radius, i.e. the rotation radius of centrifuge 100. The terms RFC, RPM, and Radius are explained and a formula for RPM is shown in the following:

$$RCF = relative centrifugal force ( g )$$

$$\begin{matrix}RPM = centrifuge speed in \\ revolutions per minute\end{matrix}$$

$$\begin{matrix}Radius = distance in mm from center of \\ centrifuge spindle to bottom of \\ Amicon device when in rotor\end{matrix}$$

$$\sqrt{\frac{RCF}{( 1.11 \times 10 ° ) ( Radius in mm )}} = RPM$$

A dashed line 704 gives an example of use of nomogram 700. Dashed line 704 discloses that in order to exceed RFC value 500 RPM value should be more than 2500 and Radius should be about 75 mm.

Numerous IVD tests are based on serum/plasma and the separation of whole blood into its components is thus required. In the prior art the centrifugation is time consuming and requires heavy laboratory equipment. Additionally, the further processing and analysis requires multiple steps where additional laboratory tasks are required. Those laboratory tasks can be avoided by using centrifuge 100.

Let as assume that centrifuge 100 rotates with a required RCF value, such as 1200 (1000-1500 is commonly used in 10 min separation) to separate the blood into components. The required RPM (revolutions per minute) can be approximated with the nomogram 700, or it can be calculated more precisely with the formula shown in the above. The required RPM with 1200 RCF is approximately 10100 when the radius of the separator is 1 cm.

FIG. 8 shows a system 800 comprising centrifuge 100. Centrifuge 100 is included in the all embodiments of system 800 but the rest of components 801-803 are optional.

A sample input device is intended for inputting the bodily fluid sample through opening 102 to sample chamber 101. A capillary pipe 801 is an example of the sample input device and pipette 307 and an injection (with a hollow needle) are other examples.

A test cassette 802 is intended for testing a component of the bodily fluid sample (the test cassette is also termed a test device in the prior art).

A component transfer device is intended for transferring the component of the bodily fluid sample into test cassette 802. Capillary pipe 801, pipette 307, and the injection are examples of the component transfer device, or generally speaking, they are examples of a part 803 for moving the bodily fluid sample or its component.

Test cassette 802 comprises a well 804 into which the component to be tested is transferred with the component transfer device. A side of well 804 includes an aperture (not shown) through which the component can enter inside test cassette 802.

Test cassette 802 further comprises an indicator 805 for indicating a test result. Indicator 805 shows to a user whether the test result is positive or negative. In addition or alternative, the indicator 805 shows to the user whether the test succeeded, i.e. the test result is reliable. The test cassettes are known devices and they have been manufactured for various purposes. Allergy tests, a pregnancy test, and an alcohol test are some examples of tests in which the test cassettes and system 800 can be used. The alcohol test is also an example of the saliva test. The invention is not limited to blood sample tests.

Centrifuge 100 may be a part of various systems 800. Centrifuge 100 and system 800 may be utilized in the IVD or POC testing but they can be utilized also in hospitals, for example. Users of systems 800 may be (health care) professionals, pharmacists, or common people. Parts of system 800 can be packed with instructions into a carton box. Centrifuge 100 may be a part of a large (health care) system that comprises a processor and a memory. In one embodiment system 800 is capable to a lab-on-chip analysis and communication through a user interface, for example, with a person taking the sample. In one embodiment system 800 is capable to provide the test results/analysis to (health care) persons, devices, or services locally, or over a network.

While the present invention has been described in connection with a number of exemplary embodiments, and implementations, the present invention is not so limited, but rather covers various modifications, and equivalent arrangements, which fall within the purview of the prospective claims.

1. A centrifuge (100) for a bodily fluid sample,
characterized in that the centrifuge comprises
a sample chamber (101) for receiving the bodily fluid sample through an opening (102) so that the bodily fluid sample rests against an inner surface (103) of the sample chamber and
a contact part (104) for coupling the sample chamber to an actuator (105), the actuator being configured to rotate the sample chamber and cause a sedimentation of the bodily fluid sample.

2. The centrifuge as claimed in claim 1, characterized in that the sample chamber includes a sedimentation basin (106) for gathering the heaviest component of the bodily fluid sample when the actuator rotates the sample chamber, the sedimentation basin locating on the utmost surface of the sample chamber from an axis of rotation (107).

3. The centrifuge as claimed in claim 2, characterized in that the sample chamber comprises a flexible material so that a distance between the sedimentation basin and the axis of rotation can vary.

4. The centrifuge as claimed in claim 1, characterized in that the sample chamber includes a bottom basin (305) into which a gravitation forces the heaviest component of the bodily fluid sample when a rotation of the sample chamber has stopped.

5. The centrifuge as claimed in claim 1, characterized in that the centrifuge comprises a stopper (303) on the opening.

6. The centrifuge as claimed in claim 1, characterized in that the sample chamber comprises a transparent material to provide a view inside the sample chamber.

7. The centrifuge as claimed in claim 1, characterized in that the sample chamber comprises a coating (405) on its inner surface.

8. The centrifuge as claimed in claim 1, characterized in that the contact part includes threads (401).

9. The centrifuge as claimed in claim 1, characterized in that the centrifuge comprises the actuator.

10. The centrifuge as claimed in claim 9, characterized in that the actuator comprises at least one of the following parts: an electric motor (601), a loadable accumulator (602).

11. A system (800) for a bodily fluid sample,
characterized in that the system includes such centrifuge (100) that comprises
a sample chamber (101) for receiving the bodily fluid sample through an opening (102) so that the bodily fluid sample rests against an inner surface (103) of the sample chamber and
a contact part (104) for coupling the sample chamber to an actuator (105), the actuator being configured to rotate the sample chamber and cause a sedimentation of the bodily fluid sample.

12. The system as claimed in claim 11, characterized in that the system comprises one of the following parts for moving the bodily fluid sample or its component: a pipette (307), a capillary pipe (801), an injection.

13. The system as claimed in claim 11, characterized in that the system comprises a test cassette (802).

14. The system as claimed in claim 13, characterized in that the test cassette comprises
a well (804) for receiving a component of the bodily fluid sample to be tested and
an indicator (805) for showing a test result.

15. The system as claimed in claims 12-14, characterized in that the at least one of the following parts is a fixed part of the system: a lancet for taking a blood sample, the test cassette, a part (803) for moving the bodily fluid sample or its component.
